(19) United States
(12) Patent Application Publication
(10) Pub. No.: US 2006/0257502 A1
(43) Pub. Date: Nov. 16, 2006

(54) A COMBINATION OF MITOCHONDRIAL NUTRIENTS FOR RELIEVING STRESS, PREVENTING AND IMPROVING STRESS-RELATED DISORDERS

(75) Inventor: Jiankang Liu, Berkeley,, CA (US)

Correspondence Address: JIANKANG LIU, 43 ROCK LANE, BERKELEY, CA, 94708, US

(73) Assignee: Jiankang Liu, Berkeley, CA (US)

(21) Appl. No.: 10/908,425

(22) Filed: May 11, 2005

(51) Int. Cl. A61K 33/06; A61K 31/573; A61K 31/714; A61K 31/685; A61K 31/525; A61K 31/51; A61K 31/4415; A61K 31/198; A61K 31/385; A61K 31/355

(52) U.S. Cl. 424/682; 514/171; 514/52; 514/251; 514/350; 514/355; 514/562; 514/78; 514/440; 514/458; 514/474; 514/546; 514/561; 514/699; 514/419; 514/276; 514/725

(57) ABSTRACT

A dietary supplement of mitochondrial nutrients is designed for relieving stress, preventing and improving stress-related disorders, such as chronic fatigue syndrome, diabetes, age-associated cognitive dysfunction and diseases (Parkinson's and Alzheimer's disease). The supplement composition has the following nutrients: B vitamins (cyanocobalamin 2-1,000 ug, thiamin 1-1,000 mg, niacin 15-2,000 mg, pyridoxine 1-1,000 mg, Pantothenate 5-150 mg, folic acid 400-40,000 ug), alpha-tocopherol 10-800 mg, ascorbic acid 50-10,000 mg, calcium 20-2,000 mg, vitamin A 200-10,000 ug, alpha-lipoic acid 100-1,000 mg, N-acetyl cysteine 100-3,000 mg, L-carnosine 100-9,000 mg, tyrosine 100-9,000 mg, vanillin 10-100 mg, phosphatidylserine 10-800 mg, resveratrol 10-50 mg, dehydroepiandrosterone 1-50 mg, and melatonin 0.1-3 mg, all of which have been individually used experimentally or clinically for relieving stress, preventing and treating age- and stress-related disorders and diseases but no combination of these compounds has been used. Many embodiments also contain at least one adjunct ingredient such as coenzyme Q 10-200 mg, acetyl-L-carnitine 100-2,000 mg, choline 50-1,000 mg, and creatine 100-2,000 mg.

### I. STATEMENT REGARDING FEDERALLY SPONSORED RESEARCH

Not applicable because this invention is a new concept derived from inventor's own experience and based on thousands of already published scientific papers available to public, not on a few experimental data.

### II. BACKGROUND

In 1994, Dr. Jiankang Liu and his colleagues, based on experimental data, published a paper in the International Journal of Stress Management entitled “Involvement of reactive oxygen species in emotional stress: A hypothesis based on the immobilization stress-induced oxidative damage and antioxidant defense changes in rat brain, and the effect of antioxidant treatment with reduced glutathione”. In 1999, Dr. Liu further modified the hypothesis with more findings in stress and oxidative stress studies and proposed an “Oxidative damage hypothesis of stress-associated aging acceleration”, published in the Neurochemical Research. In recent years, a variety of oxidative damage induced by stress has been demonstrated in the brain and other organs of several animal models. Meanwhile, protective effect of antioxidants is a current topic not only in stress-related disorders but also in other age-associated degenerative diseases such as cancer, Parkinson's and Alzheimer's disease. In his most recent review paper, Dr. Liu summarized the recent advances in stress and oxidative damage studies in supporting his theory, with a focus on the anti-stress and neuroprotective effects of some natural and nutritional antioxidants.

The Oxidative Damage Theory of Stress-associated Aging Acceleration can be illustrated in the slide presentation and summarized as: Stress may contribute to aging acceleration and age-related degenerative diseases. Stress is related to aging because 1) Aging process is accelerated by repeated exposure to stress, such as early appearance of gray hair, wrinkles, and many age-related diseases for those who under chronic stress; 2) Old organisms cannot adapt to stress as readily as younger counterparts. Therefore, stress-related or cortisol potentiated diseases are also age-related diseases. A good example is cancer. As we now know, cancer is age-related disease, it is also stress-related disease because 1) Stress related to progression of cancer: immunity suppression; 2) Stress reduction in decreasing mortality rate of cancer patients; 3) Relief or prevention of chronic stress is promising for rooting out cancer origins. The stress-/age-related disorders include, Psychological disorders: Anxiety, Loneliness, Boredom, Helplessness, Aggressiveness, Restlessness, Over-reaction, and Loss of motivation; Physical disorders: Tenseness, Stiff neck, Tired muscles, Flushing of skin, Chronic indigestion, Dizziness, Stomach aches, and Cold sweats; Behavioral disorders: Impatience, Carelessness, Sighing, Unable to laugh, Eating disorder, Forgetfulness, Sleeplessness, and Less communicative. Some examples of stress-/age-related diseases are Ulcer, Cancer, Type II diabetes, Osteoporosis, Depression, Glaucoma, Cataracts, Alzheimer's disease, Parkinson's disease, and Cardiovascular disease.

The biochemical mechanism is as follows: Stress and adaptation to stress require numerous homeostatic adjustments including hormones, neurotransmitters, oxidants, and other mediators, such as immune cytokines. The stress-induced hormones, neurotransmitters, mediators, and oxidants all have beneficial, but also harmful effects if out of balance. Therefore, the homeostasis of stress and adaptation should be governed by the hormone balance, neurotransmitter balance, mediator balance, and oxidant balance, as well as the interactions among these substances. The imbalance and the over-interaction of these balances may ultimately cause increased oxidant generation from mitochondria and oxidative damage to biomolecules. This increased oxidative damage may add to the oxidant burden associated with normal aerobic metabolism, which in itself, generates oxidants, causes accumulation of oxidative damage in mitochondria, and contributes to normal aging. Therefore, the stress-associated increase of oxidative damage may, in part, contribute to stress-associated aging acceleration and age-related neurodegenerative diseases, and anti-stress agents, including hormones, neurotransmitters, immune regulators, antioxidants, especially antioxidant mitochondrial nutrients, may be effective in delaying, preventing, and treating stress-associated aging acceleration and stress-related diseases, such as depression, chronic fatigue syndrome, digestive disorders, sleep disorders, heart diseases, cataract, obesity, diabetes, immune dysfunction, memory decline, and neurodegenerative diseases. Increasing evidence has accumulated to supports this hypothesis. For example, a clinical study by a group at the University of California at San Francisco found that stress accelerates telomere shorting, which is a biomarker of aging, suggesting that stress promotes earlier onset of age-related diseases (Epel E S, Blackburn E H, Lin J, Dhabhar F S, Adler N E, Morrow J D, Cawthon R M.: Accelerated telomere shortening in response to life stress. Proc Natl Acad Sci USA. Dec. 7, 2004;101 (49):17312-5). Another example to support this hypothesis and this claim is that a dietary supplement with most of the components in this claim (formulated to promote membrane and mitochondrial integrity, increase insulin sensitivity, reduce reactive oxygen and nitrogen species, and ameliorate inflammation) shows improving effects on memory, learning, aging and neuropathology in old transgenic mice overexpressing growth hormone (TGM) (Lemon J A, Boreham D R, Rollo C D.: A dietary supplement abolishes age-related cognitive decline in transgenic mice expressing elevated free radical processes. Exp Biol Med (Maywood). July 2003;228(7):800-10.), and extends the lifespan of both normal mice (11% increase) and transgenic mice overexpressing growth hormone (28% increase) (Jennifer A Lemon1,3, Douglas R Boreham2, C David Rollo.: A Complex Dietary Supplement Extends Longevity of Mice J. Gerontology 60A:275-9, 2005).

Dr. Jiankang Liu's publications on stress, anti-stress and stress management with mitochondrial cofactors and antioxidant nutrients:

1) Liu J K and Mori A.: Involvement of reactive oxygen species in emotional stress: A hypothesis based on the immobilization stress-induced oxidative damage in rat brain and the effect of antioxidant treatment with reduced glutathione. International Journal of Stress management 1 (3): 249-263, 1994

2) Liu J, Wang X, Mori A.: Immobilization stress-induced antioxidant defense changes in rat plasma: effect of treatment with reduced glutathione. Int J Biochem. April 1994;26(4):511-7.

3) Liu J, Wang X, Shigenaga M K, Yeo H C, Mori A, Ames B N.: Immobilization stress causes oxidative damage to lipid, protein, and DNA in the brain of rats. FASEB J. November 1996; 10(13):1532-8.

4) Jiankang Liu, Mark K. Shigenaga, Akitane Mori and Bruce N. Ames: FREE RADICALS AND NEURODEGENERATIVE DISEASES: STRESS AND OXIDATIVE DAMAGE. Free Radicals in Brain Physiology and Disorders Edited by L. Packer, M. Hiramatsu and T. Yoshikawa; Academic Press pp 403-437, 1996

5) Liu J, Yokoi I, Doniger S J, Kabuto H, Mori A, Ames B N Adrenalectomy causes oxidative damage and monoamine increase in the brain of rats and enhances immobilization stress-induced oxidative damage and neurotransmitter changes. INTERNATIONAL JOURNAL OF STRESS MANAGEMENT 5 (1): 39-56 January 1998

6) Liu J, Mori A.: Stress, aging, and brain oxidative damage. Neurochem Res. November 1999;24(11):1479-97

7) Jiankang Liu, Xiaoyan Wang and Akitane Mori: Antioxidant Mechanism of the Anti-stress Effects of Medicinal Herbs: A Biochemical, Pharmacological, and Toxicological Study. Molecular Aspects of Asian Medicine, edited by A. Mori and T. Satoh, PJD Publications Limited, New York pp 355-386, 2001

8) Zhongliang Zhu, Weina Chen, Xia Li, Hui Li, Xinlin Chen, Bin Tang, Zhuanli Bai, Ning JiaJiankang Liu: Prenatal stress causes gender-dependent neuronal loss and oxidative stress in the hippocampus of rats. J. Neurosci. Res. 78:837-844, 2004

9) Jiankang Liu and Akitane Mori: Oxidative Damage Hypothesis of stress-associated aging acceleration: neuroprotective effects of natural and nutritional antioxidants. Res. Commun Brain & Nerve (in press) 2005

10) Jiankang Liu and Bruce N. Ames: Reducing mitochondrial decay with mitochondrial nutrients to delay and treat cognitive dysfunction, Alzheimer's and Parkinson's disease. Nutritional Neuroscience (in press), 2005.

### III. SUMMARY OF THE INVENTION

The scientific basis of this combination is our oxidative damage hypothesis of stress-associated aging acceleration and disease. We propose that stress accelerates aging process (makes people old faster) through a mechanism of increasing oxidative metabolism; the increase oxidative stress/damage is the major cause of stress-related and age-related disorders and diseases, such as Ulcer, Cancer, Type II diabetes, Osteoporosis, Depression, Glaucoma, Cataracts, Alzheimer's disease, Parkinson's disease, and Cardiovascular disease. The mechanism can be simplified as follows: Stress and adaptation to stress require numerous homeostatic adjustments of the balance of hormones, neurotransmitters, oxidants, and other mediators. Stress-induced imbalance and the over-interaction of these balances may ultimately cause increased oxidant generation in the mitochondria, causing oxidative damage to lipids, proteins, and nucleic acids, weakening antioxidant defense systems, and further affecting the homeostasis of numerous mediators of defense reactions including hormones, neurotransmitters, and immune cytokines, all of which, like oxidants, are dangerous when losing balance. This increased oxidative damage may add to the oxidant burden associated with normal aerobic metabolism, which in itself, generates oxidants, causes accumulation of oxidative damage in mitochondria, and contributes to normal aging. Therefore, the stress-associated increase of oxidative damage may, in part, contribute to stress-associated aging acceleration and age-related neurodegenerative diseases. A variety of oxidative damage induced by stress has been demonstrated in the brain and other organs of several animal models, as well as in clinical observations to support this hypothesis. Meanwhile, protective effects of nutrients and antioxidants have been reported in stress and stress- and age-related disorders and diseases. In short, we provide evidence that mitochondrial decay strongly associates with stress and stress-/age-associated disorders and diseases, and that all of ingredients in this composition can work in different ways for preventing, recovering, and repairing mitochondrial damage and improving mitochondrial function, thus, relieving stress, preventing and improving stress-/age-related disorders and diseases. In addition, none of these nutrients are known to have toxicities or side effects in doses that are likely to be therapeutically beneficial in this claim.

The composition will be consisted with the following preferred forms of the following natural mitochondrial cofactors and nutrients: cyanocobalamin, thiamin, niacin, pyridoxine, Pantothenate, folic acid, alpha-tocopherol, ascorbic acid, calcium, vitamin A, alpha-lipoic acid, N-acetyl cysteine, L-carnosine, tyrosine, vanillin, phosphatidylserine, resveratrol, DHEA, and melatonin.

Embodiment: A method of relieving stress and preventing and improving stress-related disorders has the step of administering effective amounts of cyanocobalamin, thiamin, niacin, pyridoxine, Pantothenate, folic acid, alpha-tocopherol, ascorbic acid, calcium, vitamin A, alpha-lipoic acid, N-acetyl cysteine, L-carnosine, tyrosine, vanillin, phosphatidylserine, resveratrol, dehydroepiandrosterone, and melatonin. Many embodiments also contain at least one adjunct ingredient such as coenzyme Q, acetyl-L-carnitine, choline, and creatine. Since the mitochondrial nutrients and natural metabolites cited here are safe and affordable, a composition of dietary supplementation with these nutrients may prove to be a practical strategy for relieving stress, and prevention, and improvement/management of stress-related disorders. There are no drugs able to completely prevent or cure stress-related disorders. None of the current drugs has been very effective. In addition, drugs are expensive and require regular physician monitoring to avoid potentially dangerous side effects, would appear to be less practical options from cost-effectiveness, convenience and safety standpoints for stress management and stress-related disorder treatment. Given the fact that the population at stress and at-risk for stress-related disorders is huge, there exists a quite big market.

### IV DETAILED DESCRIPTION

The rationale for using the substances, the optimal doses, and for mixing of them in the embodiment is detailed below, based on published studies with cells, animals, and clinical trials on the mitochondrial nutrients for relieving stress, preventing and improving stress-/age-related disorders and degenerative diseases.

1. Vitamins (Alpha-Tocopherol-VE, Ascorbic Acid-VC, Vitamin A/Beta-Carotene Cyanocobalamin-B12, Thiamin-B1, Niacin-B3, Pyridoxine-B6, Pantothenate-B5, Folic Acid).

The vitamins have been widely used as a dietary supplements. This patent application uses these vitamins based on their function as mitochondrial cofactors and nutrients, and more importantly with a relative higher doses (several to 100 fold of their dietary reference intakes (DRIs, recommended dietary allowance RDA or Adequate Intake (AI)) to prevent and reduce stress-induced oxidative mitochondrial decay, leading to stress relief and prevention and improvement of stress-related disorders. Studies have shown:

These vitamins are working with unique functions in mitochondria to prevent and reduce mitochondrial decay. For example, the B vitamins are precursors of different mitochondrial enzyme cofactors (thiamine is the precursor of coenzyme thiamine pyrophosphate; riboflavin is the precursor of flavin mononucleotide (FMN) and flavin adenine dinucleotide (FAD); niacin is the precursor of nicotinamide adenine dinucleotide (NAD) and nicotinamide adenine dinucleotide phosphate (NADP); pantothenate is a component of coenzyme A; pyridoxine is the precursor of pyridoxal phosphate; and biotin is the cofactor of many carboxylases); vitamin K in combination with vitamin C serves as electron acceptors to bypass a deficiency in complex III (Price, M C. Longevity Report 91, www.quantium.cwc.net/lr91.html, 2001; Marriage B, Clandinin M T, Glerum D M.: Nutritional cofactor treatment in mitochondrial disorders. J Am Diet Assoc. August 2003;103(8):1029-38; Liu, J and Ames, B N.: Reducing mitochondrial decay with mitochondrial nutrients to delay and treat cognitive dysfunction, Alzheimer's and Parkinson's disease. Nutr. Neurosci. in press, 2005)

Deficiency causes and supplementation prevents oxidative stress, mitochondrial decay, and stress-/age-related disease (Liu, J and Ames, B N.: Reducing mitochondrial decay with mitochondrial nutrients to delay and treat cognitive dysfunction, Alzheimer's and Parkinson's disease. Nutr. Neurosci. in press, 2005)

Vitamins reduce stress-induced mitochondrial decay, oxidant generation, thus relieve stress, and prevent and ameliorate stress-related diseases (Liu J, Mori A.: Stress, aging, and brain oxidative damage. Neurochem Res. November 1999;24(11):1479-97; Jiankang Liu and Akitane Mori: Oxidative damage hypothesis of stress-associated aging acceleration: neuroprotective effects of natural and nutritional antioxidants. Res. Commun Brain & Nerve (in press) 2005).

Vitamin E is shown to preventing stress-induced oxidative damage and gastric ulceration as an anti-stress agent (Das D, Bandyopadhyay D, Bhattacharjee M, Banerjee R K. Hydroxyl radical is the major causative factor in stress-induced gastric ulceration. Free Radic Biol Med. 1997;23(1):8-18).

More importantly, high doses (supraphysiological doses) can also ameliorate genetic diseases (Ames B N, Elson-Schwab I, Silver E A.: High-dose vitamin therapy stimulates variant enzymes with decreased coenzyme binding affinity (increased K(m)): relevance to genetic disease and polymorphisms. Am J Clin Nutr. April 2002;75(4):616-58).

2. alpha-Lipoic Acid

alpha-Lipoic acid is a naturally occurring compound that is synthesized by plants and animals, including humans, and functions as a cofactor for several important enzymes as well as a potent antioxidant. Studies have shown the following effects on stress and stresss-related disorders:

Regulation of the neurohumoral systems of patients with ischemic heart disease and under emotional-pain stress (Fomichev V I, Pchelintsev V P.: The neurohumoral systems of patients with ischemic heart disease and under emotional-pain stress: the means for their pharmacological regulation. Kardiologiia. 1993;33(10):15-8).

Lifespan-extending effect in Drosophila melanogaster (Bauer J H, Goupil S, Garber G B, Helfand S L.: An accelerated assay for the identification of lifespan-extending interventions in Drosophila melanogaster. Proc Natl Acad Sci USA. August 2004 31 ;101 (35):12980-5).

Restoration of the antibody response in immunosuppressed mice (Ohmori H, Yamauchi T, Yamamoto I.: Augmentation of the antibody response by lipoic acid in mice. II. Restoration of the antibody response in immunosuppressed mice. Jpn J Pharmacol. October 1986;42(2):275-80).

Preventing cognitive impairment and oxidative stress induced by intracerebroventricular streptozotocin in rats (Sharma M, Gupta Y K.: Effect of alpha lipoic acid on intracerebroventricular streptozotocin model of cognitive impairment in rats. Eur Neuropsychopharmacol. August 2003;13(4):241 -7).

Reversal of memory impairment and brain oxidative stress in aged SAMP8 mice (Farr S A, Poon H F, Dogrukol-Ak D, Drake J, Banks W A, Eyerman E, Butterfield D A, Morley J E.: The antioxidants alpha-lipoic acid and N-acetylcysteine reverse memory impairment and brain oxidative stress in aged SAMP8 mice. J Neurochem. March 2003;84(5):1173-83).

Partial reversal of memory loss, brain mitochondrial decay, and RNA/DNA oxidation in old rats (Liu J, Head E, Gharib A M, Yuan W, Ingersoll R T, Hagen T M, Cotman C W, Ames B N.: Memory loss in old rats is associated with brain mitochondrial decay and RNA/DNA oxidation: partial reversal by feeding acetyl-L-carnitine and/or R-alpha-lipoic acid. Proc Natl Acad Sci USA. February 2002 19;99(4):2356-61).

Reversal of functional deficits in the peripheral and central nervous system of streptozotocin-diabetic rats (Biessels G J, Smale S, Duis S E, Kamal A, Gispen W H.: The effect of gamma-linolenic acid-alpha-lipoic acid on functional deficits in the peripheral and central nervous system of streptozotocin-diabetic rats. J Neurol Sci. January 2001 1;182(2):99-106).

Improving memory in aged mice (Stoll S, Hartmann H, Cohen S A, Muller W E.: The potent free radical scavenger alpha-lipoic acid improves memory in aged mice: putative relationship to NMDA receptor deficits. Pharmacol Biochem Behav. December 1993;46(4):799-805).

Treatment for depression (Salazar M R.: Alpha lipoic acid: a novel treatment for depression. Med Hypotheses. December 2000;55(6):510-2).

Treatment for diabetic neuropathy (Morelli V, Zoorob R J.: Alternative therapies: Part I. Depression, diabetes, obesity. Am Fam Physician. Sep. 1, 2000;62(5):1051-60).

Clinical treatments for insulin resistance in type II diabetes, retinopathy, cataract, glaucoma, HIV/AIDS, cancer, liver disease, Wilson's disease, cardiovascular disease, lactic acidosis, Alzheimer type dementia (Liu J, Atamna H, Kuratsune H, Ames B N.: Delaying brain mitochondrial decay and aging with mitochondrial antioxidants and metabolites. Ann N Y Acad Sci. April 2002;959:133-66).

3. GSH/N-acetyl-cysteine

Glutathione (GSH) N-acetyl-cysteine are one of the most important natural small molecular antioxidants in human body. Studies have shown they have the following effects on stress and stress-related disorders:

Preventing stress-induced oxidative damage and gastric ulceration as an anti-stress agent (Das D, Bandyopadhyay D, Bhattacharjee M, Banerjee R K. Hydroxyl radical is the major causative factor in stress-induced gastric ulceration. Free Radic Biol Med. 1997;23(1):8-18).

Reducing emotional stress-induced oxidative damage in the brain of rats (Liu J K and Mori A.: Involvement of reactive oxygen species in emotional stress: A hypothesis based on the immobilization stress-induced oxidative damage in rat brain and the effect of antioxidant treatment with reduced glutathione. International Journal of Stress management 1 (3): 249-263, 1994).

Reducing ulcer and enhancing blood antioxidant defense in rats (Liu J, Wang X, Mori A.: Immobilization stress-induced antioxidant defense changes in rat plasma: effect of treatment with reduced glutathione. INT J BIOCHEM. April 1994;26(4):511-7).

Playing important role in against stress-induced oxidative damage to lipid, protein, and DNA in the brain of rats (Liu J, Wang X, Shigenaga M K, Yeo H C, Mori A, Ames B N. Immobilization stress causes oxidative damage to lipid, protein, and DNA in the brain of rats. FASEB J. November 1996; 10(13):1532-8).

Beneficial effects on chronic fatigue syndrome (Logan A C, Wong C. Chronic fatigue syndrome: oxidative stress and dietary modifications. Altern Med Rev. October 2001;6(5):450-9).

Improving cardiac performance in patients with septic shock (Peake S L, Moran J L, Leppard P I. N-acetyl-L-cysteine depresses cardiac performance in patients with septic shock. Crit Care Med. August 1996;24(8):1302-10).

Reversing memory impairment and brain oxidative stress in aged senescence accelerated mice (Farr S A, Poon H F, Dogrukol-Ak D, Drake J, Banks W A, Eyerman E, Butterfield D A, Morley J E.: The antioxidants alpha-lipoic acid and N-acetylcysteine reverse memory impairment and brain oxidative stress in aged SAMP8 mice. J Neurochem. March 2003;84(5):1173-83).

4. L-Carnosine

Carnosine, homocarnosine, and anserine are present in high concentrations in the muscle and brain of many animals and humans. Studies have shown that L-carnosine has the following functions and effects on stress and stress-related disorders:

Antioxidant activity (Kohen R, Yamamoto Y, Cundy K C, Ames B N.: Antioxidant activity of carnosine, homocarnosine, and anserine present in muscle and brain. Proc Natl Acad Sci USA. May 1988;85(9):3175-9; Klebanov G I, Teselkin Yu O, Babenkova I V, Lyubitsky O B, Rebrova O Yu, Boldyrev A A, Vladimirov Yu A.: Effect of carnosine and its components on free-radical reactions. Membr Cell Biol. 1998;12(1):89-99).

The SOD like antioxidant activity (Kohen R, Misgav R, Ginsburg I.: The SOD like activity of copper:carnosine, copper:anserine and copper:homocarnosine complexes. Free Radic Res Commun. 1991;12-13 Pt 1:179-85).

Neuroprotective effects on pheochromocytoma PC12 cells exposed to ischemia (Tabakman R, Lazarovici P, Kohen R.: Neuroprotective effects of carnosine and homocarnosine on pheochromocytoma PC12 cells exposed to ischemia. J Neurosci Res. May 15, 2002;68(4):463-9; Tabakman R, Jiang H, Levine R A, Kohen R, Lazarovici P.: Apoptotic characteristics of cell death and the neuroprotective effect of homocarnosine on pheochromocytoma PC12 cells exposed to ischemia. J Neurosci Res. Feb. 15, 2004;75(4):499-507).

Protective effect on free radical lipid oxidation during acute stress in rats (Guliaeva N V, Obidin A B, Levshina I P, Filonenko A V, Dupin A M, Boldyrev A A.: The effect of carnosine on indicators of free radical lipid oxidation during acute stress in rats. Nauchnye Doki Vyss Shkoly Biol Nauki. 1989(8):5-16).

Protective effects on acute or chronic gastric lesions and gastric secretion in rat (Kunimi H, Okabe S.: Effects of CL-1700 and its constituents on acute or chronic gastric lesions and gastric secretion in rats. Jpn J Pharmacol. June 1982;32(3):469-77).

Protective effect on Cu,Zn-superoxide dismutase during impaired oxidative metabolism in the brain (Stvolinskii S L, Fedorova T N, Yuneva M O, Boldyrev A A.: Protective effect of carnosine on Cu,Zn-superoxide dismutase during impaired oxidative metabolism in the brain in vivo. Bull Exp Biol Med. February 2003;135(2):130-2).

Protecting rat cerebellar granular cells from free radical damage (Boldyrev A A, Johnson P, Wei Y, Tan Y, Carpenter D O.: Carnosine and taurine protect rat cerebellar granular cells from free radical damage. Neurosci Lett. Mar. 26, 1999;263(2-3):169-72).

Antioxidant, antihypertensive, immunomodulating, wound healing, and antineoplastic effects (Quinn P J, Boldyrev A A, Formazuyk V E.: Carnosine: its properties, functions and potential therapeutic applications. Mol Aspects Med. 1992;13(5):379-444).

Accelerating metabolism of stress-related substances in rats (Nagai K, Suda T, Kawasaki K, Yamaguchi Y.: Acceleration of metabolism of stress-related substances by L-carnosine. Nippon Seirigaku Zasshi. 1990;52(7):221-8).

Prevents the activation of free-radical lipid oxidation during stress (Guliaeva N V, Dupin A M, Levshina I P, Obidin A B, Boldyrev A A.: Carnosine prevents the activation of free-radical lipid oxidation during stress. Biull Eksp Biol Med. February 1989;107(2):144-7).

Therapeutic tool to manage age-related cataracts in human and in canine eyes (Babizhayev M A, Deyev A I, Yermakova V N, Brikman I V, Bours J. Lipid peroxidation and cataracts: N-acetylcarnosine as a therapeutic tool to manage age-related cataracts in human and in canine eyes. Drugs R D. 2004;5(3):125-39).

5. Tyrosine

Tyrosine is a precursor of neurotransmitter norepinephrine. Brain norepinephrine levels are closely related to stress-induced performance decrement. Therefore, supplementation of tyrosine may be useful in counteracting any stress-related performance decrement and mood deterioration in the following way: First, various forms of stress induce brain depletion of catecholamines, especially norepinephrine, in animals. Second, brain norepinephrine levels are closely related to stress-induced performance decrement in animals. Third, the administration of tyrosine may minimize or reverse stress-induced performance decrement by increasing depleted brain norepinephrine levels. Studies have shown that tyrosine has the following functions and effects on stress and stress-related disorders:

An aid to stress resistance among troops (Salter C A.: Dietary tyrosine as an aid to stress resistance among troops. Mil Med. March 1989;154(3):144-6).

Improving mood and reversing stress-induced performance decrement in army (Owasoyo J O, Neri D F, Lamberth J G: Tyrosine and its potential use as a countermeasure to performance decrement in military sustained operations. Aviat Space Environ Med. May 1992;63(5):364-9).

Counteracting performance decrements during episodes of sustained work coupled with sleep loss in army (Neri D F, Wiegmann D, Stanny R R, Shappell S A, McCardie A, McKay D L.: The effects of tyrosine on cognitive performance during extended wakefulness. Aviat Space Environ Med. April 1995;66(4):313-9).

Improves cognitive performance and reducing blood pressure in cadets after one week of a combat training course (Deijen J B, Wientjes C J, Vullinghs H F, Cloin P A, Langefeld J J.: Tyrosine improves cognitive performance and reduces blood pressure in cadets after one week of a combat training course. Brain Res Bull. Jan. 15, 1999;48(2):203-9).

Reversing a cold-induced working memory deficit in humans (Shurtleff D, Thomas J R, Schrot J, Kowalski K, Harford R.: Tyrosine reverses a cold-induced working memory deficit in humans. Pharmacol Biochem Behav. April 1994;47(4):935-41).

Reducing environmental stress in humans (Banderet L E, Lieberman H R.: Treatment with tyrosine, a neurotransmitter precursor, reduces environmental stress in humans. Brain Res Bull. April 1989;22(4):759-62).

Improving cognitive function and regulating blood pressure under stress in humans (Deijen J B, Orlebeke J F.: Effect of tyrosine on cognitive function and blood pressure under stress. Brain Res Bull. 1994;33(3):319-23).

Improves working memory in a multitasking environment in humans (Thomas J R, Lockwood P A, Singh A, Deuster P A.: Tyrosine improves working memory in a multitasking environment. Pharmacol Biochem Behav. November 1999;64(3):495-500).

Ameliorating some effects of lower body negative pressure stress in humans (Dollins A B, Krock L P, Storm W F, Wurtman R J, Lieberman H R.: L-tyrosine ameliorates some effects of lower body negative pressure stress. Physiol Behav. February 1995;57(2):223-30).

Preventing hypoxia-induced decrements in learning and memory (Shukitt-Hale B, Stillman M J, Lieberman H R.: Tyrosine administration prevents hypoxia-induced decrements in learning and memory. Physiol Behav. April-May 1996;59(4-5):867-71).

Ameliorating a cold-induced delayed matching-to-sample performance decrement in rats (Shurtleff D, Thomas J R, Ahlers S T, Schrot J.: Tyrosine ameliorates a cold-induced delayed matching-to-sample performance decrement in rats. Psychopharmacology (Berl). 1993;112(2-3):228-32).

Playing a role in chronic fatigue syndrome (CFS): lower leveling brain is correlated to CFS (Georgiades E, Behan W M, Kilduff L P, Hadjicharalambous M, Mackie E E, Wilson J, Ward S A, Pitsiladis Y P.: Chronic fatigue syndrome: new evidence for a central fatigue disorder. Clin Sci (Lond). August 2003;105(2):213-8).

6. Vanilline

Vanilline is a natural component and has long been used as a food additive. More recent studies demonstrate that vanilline has the following functions and effects on stress and stress-related disorders:

Antioxidant activities: Effects on free radicals, brain peroxidation and degradation of benzoate, deoxyribose, amino acids and DNA (Liu J, Mori A.: Antioxidant and pro-oxidant activities of p-hydroxybenzyl alcohol and vanillin: effects on free radicals, brain peroxidation and degradation of benzoate, deoxyribose, amino acids and DNA. Neuropharmacology. July 1993;32(7):659-69; Aruoma O I.: Antioxidant actions of plant foods: use of oxidative DNA damage as a tool for studying antioxidant efficacy. Free Radic Res. June 1999;30(6):419-27).

Antimutagenic, anticlastogenic and anticarcinogenic actions (Durant S, Karran P.: Vanillins—a novel family of DNA-PK inhibitors. Nucleic Acids Res. Oct. 1, 2003;31(19):5501 -12)

A positive foreground stimulus of the acoustic startle reflex in humans (Miltner W, Matjak M, Braun C, Diekmann H, Brody S.: Emotional qualities of odors and their influence on the startle reflex in humans. Psychophysiology. January 1994;31(1):107-10).

7. Phosphatidylserine

Studies have shown that phosphatidylserine has the following functions and effects on stress and stress-related disorders:

Beneficial effects on the neuroendocrine response to physical stress in humans (Monteleone P, Beinat L, Tanzillo C, Maj M, Kemali D.: Effects of phosphatidylserine on the neuroendocrine response to physical stress in humans. Neuroendocrinology. September 1990;52(3):243-8).

Blunting stress-induced activation of the hypothalamo-pituitary-adrenal axis in healthy men (Monteleone P, Maj M, Beinat L, Natale M, Kemali D.: Blunting by chronic phosphatidylserine administration of the stress-induced activation of the hypothalamo-pituitary-adrenal axis in healthy men. Eur J Clin Pharmacol. 1992;42(4):385-8.)

8. Resveratrol

Resveratrol, trans-3,5,4′-trihydroxystilbene, was first isolated in 1940 as a constituent of the roots of white hellebore (Veratrum grandiflorum O. Loes), but has since been found in various plants, including grapes, berries, peanuts and red wine. Studies have shown it the following functions and effects on stress and stress-related disorders:

Preventing and improving heart disease and cancer in humans (Aggarwal B B, Bhardwaj A, Aggarwal R S, Seeram N P, Shishodia S, Takada Y. Role of resveratrol in prevention and therapy of cancer: preclinical and clinical studies. Anticancer Res. September-October 2004;24(5A):2783-840; Aziz M H, Kumar R, Ahmad N.: Cancer chemoprevention by resveratrol: in vitro and in vivo studies and the underlying mechanisms (review). Int J Oncol. July 2003;23(1):17-28).

Delaying ageing in metazoans (Wood J G, Rogina B, Lavu S, Howitz K, Helfand S L, Tatar M, Sinclair D.: Sirtuin activators mimic caloric restriction and delay ageing in metazoans. Nature. Aug. 5, 2004;430(7000):686-9).

Extending Saccharomyces cerevisiae lifespan (Howitz K T, Bitterman K J, Cohen H Y, Lamming D W, Lavu S, Wood J G, Zipkin R E, Chung P, Kisielewski A, Zhang L L, Scherer B, Sinclair D A.: Small molecule activators of sirtuins extend Saccharomyces cerevisiae lifespan. Nature. Sep. 11, 2003;425(6954):191-6).

Lifespan-extending effect in Drosophila melanogaster (Bauer J H, Goupil S, Garber G B, Helfand S L.: An accelerated assay for the identification of lifespan-extending interventions in Drosophila melanogaster. Proc Natl Acad Sci USA. Aug. 31, 2004;101 (35):12980-5).

Suppressing the angiogenesis and tumor growth of gliomas in rats (Tseng S H, Lin S M, Chen J C, Su Y H, Huang H Y, Chen C K, Lin P Y, Chen Y.: Resveratrol suppresses the angiogenesis and tumor growth of gliomas in rats. Clin Cancer Res. Mar. 15, 2004;10(6):2190-202).

Suppressing 7,12-dimethylbenz(a)anthracene-induced mammary carcinogenesis in rats (Banerjee S, Bueso-Ramos C, Aggarwal B B.: Suppression of 7,12-dimethylbenz(a)anthracene-induced mammary carcinogenesis in rats by resveratrol: role of nuclear factor-kappaB, cyclooxygenase 2, and matrix metalloprotease 9. Cancer Res. Sep. 1, 2002;62(17):4945-54).

Suppressing N-nitrosomethylbenzylamine (NMBA)-induced esophageal tumorigenesis in F344 rats (Li Z G, Hong T, Shimada Y, Komoto I, Kawabe A, Ding Y, Kaganoi J, Hashimoto Y, Imamura M.: Suppression of N-nitrosomethylbenzylamine (NMBA)-induced esophageal tumorigenesis in F344 rats by resveratrol. Carcinogenesis. September 2002;23(9):1531-6).

Inhibiting cell proliferation and preventing oxidative DNA damage (Sgambato A, Ardito R, Faraglia B, Boninsegna A, Wolf F I, Cittadini A.: Resveratrol, a natural phenolic compound, inhibits cell proliferation and prevents oxidative DNA damage. Mutat Res. Sep. 20, 2001;496(1-2):171-80).

Suppressing hepatoma cell invasion (Kozuki Y, Miura Y, Yagasaki K.: Resveratrol suppresses hepatoma cell invasion independently of its anti-proliferative action. Cancer Lett. Jun. 26, 2001;167(2):151-6).

Preventing intracerebroventricular streptozotocin induced cognitive impairment and oxidative stress in rats (Sharma M, Gupta Y K.: Chronic treatment with trans resveratrol prevents intracerebroventricular streptozotocin induced cognitive impairment and oxidative stress in rats. Life Sci. Oct. 11, 2002;71(21):2489-98).

Protecting neurons from beta-amyloid neurotoxicity (Savaskan E, Olivieri G, Meier F, Seifritz E, Wirz-Justice A, Muller-Spahn F.: Red wine ingredient resveratrol protects from beta-amyloid neurotoxicity. Gerontology. November-December 2003;49(6):380-3).

Protecting lipoprotein oxidition in neuronal cell death: implication for preventing neurodegeneration (Draczynska-Lusiak B, Doung A, Sun A Y.: Oxidized lipoproteins may play a role in neuronal cell death in Alzheimer disease. Mol Chem Neuropathol. February 1998;33(2):139-48).

Daily oral administration of high doses to is not harmful (Juan M E, Vinardell M P, Planas J M.: The daily oral administration of high doses of trans-resveratrol to rats for 28 days is not harmful. J Nutr. February 2002;132(2):257-60).

No observed adverse effect level was 300 mg per kilogram body weight per day in rats (Crowell J A, Korytko P J, Morrissey R L, Booth T D, Levine B S.: Resveratrol-associated renal toxicity. Toxicol Sci. December 2004;82(2):614-9).

9. Dehydroepiandrosterone (DHEA)

DHEA has long been considered an anti-stress hormone. Studies have demonstrate that DHEA has the following functions and effects on stress and stress-related disorders:

Anti-stress and antioxidant effects (Hu Y, Cardounel A, Gursoy E, Anderson P, Kalimi M.: Anti-stress effects of dehydroepiandrosterone: protection of rats against repeated immobilization stress-induced weight loss, glucocorticoid receptor production, and lipid peroxidation. Biochem Pharmacol. Apr. 1, 2000;59(7):753-62).

Anti-stress response and effects on anxiety and cortisol metabolism (Boudarene M, Legros J J, Timsit-Berthier M.: Study of the stress response: role of anxiety, cortisol and DHEAs. Encephale. March-April 2002;28(2):139-46).

Higher level is important for management on stress, emotions, heart rate variability, hormone metabolism (McCraty R, Barrios-Choplin B, Rozman D, Atkinson M, Watkins A D.: The impact of a new emotional self-management program on stress, emotions, heart rate variability, DHEA and cortisol. Integr Physiol Behav Sci. April-June 1998;33(2):151-70).

Enhancing memory-related hippocampal plasticity under stress (Diamond D M, Fleshner M, Rose G M.: The enhancement of hippocampal primed burst potentiation by dehydroepiandrosterone sulfate (DHEAS) is blocked by psychological stress. Stress. December 1999;3(2):107-21).

Protecting memory-related hippocampal cells from oxidative stress-induced damage (Bastianetto S, Ramassamy C, Poirier J, Quirion R.: Dehydroepiandrosterone (DHEA) protects hippocampal cells from oxidative stress-induced damage. Brain Res Mol Brain Res. Mar. 20, 1999;66(1-2):35-41).

Chronic fatigue syndrome is associated with deficiency (Kuratsune H, Yamaguti K, Sawada M, Kodate S, Machii T, Kanakura Y, Kitani T.: Dehydroepiandrosterone sulfate deficiency in chronic fatigue syndrome. Int j Mol Med. January 1998; 1(1): 143-6).

Neurosteroids: of the nervous system, by the nervous system, for the nervous system (Baulieu E E.: Neurosteroids: of the nervous system, by the nervous system, for the nervous system. Recent Prog Horm Res. 1997;52:1-32).

As a memory enhancer: effects on central neurons and implications for psychiatric and neurological disorders (Holsboer F, Grasser A, Friess E, Wiedemann K.: Steroid effects on central neurons and implications for psychiatric and neurological disorders. Ann N Y Acad Sci. Nov. 30, 1994;746:345-59; discussion 359-61; DHEA. Monograph. Altern Med Rev. June 2001;6(3):314-8).

10. Melatonin

Melatonin (N-acetyl-5-methoxytryptamine), originally discovered in the pineal gland, is now known also to be present in the gastrointestinal tract from the stomach to the colon. Studies have demonstrate that melatoni has the following functions and effects on stress and stress-related disorders:

Relieves the Neural Oxidative Burden that Contributes to Dementias (Reiter R J, Tan D X, Pappolla M A.: Melatonin Relieves the Neural Oxidative Burden that Contributes to Dementias. Ann N Y Acad Sci. December 2004;1035:179-96).

Beneficial actions in experimental models of stroke (Reiter R J, Tan D X, Leon J, Kilic U, Kilic E.: When melatonin gets on your nerves: its beneficial actions in experimental models of stroke. Exp Biol Med (Maywood). February 2005;230(2):104-17).

Antioxidant effects and protection to neurological damage (Reiter R J.: Melatonin, active oxygen species and neurological damage. Drug News Perspect. June 1998;11(5):291-6).

Reducing prostate cancer cell growth leading to neuroendocrine differentiation (Sainz R M, Mayo J C, Tan D X, Leon J, Manchester L, Reiter R J.: Melatonin reduces prostate cancer cell growth leading to neuroendocrine differentiation via a receptor and PKA independent mechanism. Prostate. Apr. 1, 2005;63(1):29-43).

Detoxification of oxygen and nitrogen-based toxic reactants (Reiter R J, Tan D X, Manchester L C, Lopez-Burillo S, Sainz R M, Mayo J C. Melatonin: detoxification of oxygen and nitrogen-based toxic reactants. Adv Exp Med Biol. 2003;527:539-48).

Mitigating mitochondrial malfunction (Josefa León1, Darío Acuña-Castroviejo2, Germane Escames2, Dun-Xian Tan1 and Russel J. Reiter.: Melatonin mitigates mitochondrial malfunction. J Pineal Res. January 2005;38(1):1-9).

Preserving the integrity of the mitochondria and reducing mitochondrial dysfunction (Leon J, Acuna-Castroviejo D, Sainz R M, Mayo J C, Tan D X, Reiter R J. Melatonin and mitochondrial function. Life Sci. Jul. 2, 2004;75(7):765-90)

Beneficial effects on oxidative and inflammatory parameters in respiratory distress syndrome of preterm newborns (Gitto E, Reiter R J, Cordaro S P, La Rosa M, Chiurazzi P, Trimarchi G, Gitto P, Calabro M P, Barberi I.: Oxidative and inflammatory parameters in respiratory distress syndrome of preterm newborns: beneficial effects of melatonin. Am J Perinatol. May 2004;21 (4):209-16)

Beneficial actions in the gastrointestinal tract to form ulcer (Reiter R J, Tan D X, Mayo J C, Sainz R M, Leon J, Bandyopadhyay D.: Neurally-mediated and neurally-independent beneficial actions of melatonin in the gastrointestinal tract. J Physiol Pharmacol. December 2003;54 Suppl 4:113-25).

Protecting the behavioural disturbances induced by chronic mild stress in mice (Kopp C, Vogel E, Rettori M C, Delagrange P, Misslin R.: The effects of melatonin on the behavioural disturbances induced by chronic mild stress in C3H/He mice. Behav Pharmacol. February 1999;10(1):73-83).

Counteracting stress hormone glucocorticoid-induced dysregulation of the hypothalamic-pituitary-adrenal axis in the rat (Konakchieva R, Mitev Y, Almeida O F, Patchev V K. Chronic melatonin treatment counteracts glucocorticoid-induced dysregulation of the hypothalamic-pituitary-adrenal axis in the rat. Neuroendocrinology. March 1998;67(3):171-80).

Exogenous melatonin is consumed against emotional stress (Malinovskaia N K, Pertsov S S, Sosnovskii A S, Vetterberg L, Friberg I, Voznesenskaia L A, Rapoport S I, Komarov F I, Sudakov K V.: Emotional stress and blood melatonin levels. Vestn Ross Akad Med Nauk. 1997(7):51-4).

Inhibitory effects on the central nevous system, on sensory reflex and on stress responses (Datta P C, Hoehler F K, Sandman C A.: Effects of melatonin on startle reflex in rat. Peptides. 1981;2 Suppl 1:155-60).

Reducing memory loss and neural oxidative damage in an aging accelerating mice model (Shen Y X, Xu S Y, Wei W, Sun X X, Yang J, Liu L H, Dong C.: Melatonin reduces memory changes and neural oxidative damage in mice treated with D-galactose. J Pineal Res. April 2002;32(3):173-8).

Protecting rat brain from memory loss and oxidative damage induced by amyloid beta-peptide 25-35 (Shen Y X, Xu S Y, Wei W, Sun X X, Liu L H, Yang J, Dong C.: The protective effects of melatonin from oxidative damage induced by amyloid beta-peptide 25-35 in middle-aged rats. J Pineal Res. March 2002;32(2):85-9).

Protecting learning, memory and oxidative deficiencies induced by intracere-broventricular streptozotocin in rats (Sharma M, Gupta Y K.: Effect of chronic treatment of melatonin on learning, memory and oxidative deficiencies induced by intracerebroventricular streptozotocin in rats. Pharmacol Biochem Behav. October-November 2001; 70(2-3):325-31).

Reversal of aging and chronic ethanol-induced memory decay (Raghavendra V, Kulkarni S K.: Possible antioxidant mechanism in melatonin reversal of aging and chronic ethanol-induced amnesia in plus-maze and passive avoidance memory tasks. Free Radic Biol Med. Mar. 15, 2001;30(6):595-602).

Neuroimmunoregulatory, anti-stress, and anti-aging effects (Pierpaoli W, Maestroni G J.: Melatonin: a principal neuroimmunoregulatory and anti-stress hormone: its anti-aging effects. Immunol Lett. December 1987;16(3-4):355-61).

Beneficial effects on chronic fatigue syndrome in mice (Singh A, Garg V, Gupta S, Kulkarni S K.: Role of antioxidants in chronic fatigue syndrome in mice. Indian J Exp Biol. November 2002;40(11):1240-4; Singh A, Naidu P S, Gupta S, Kulkarni S K.: Effect of natural and synthetic antioxidants in a mouse model of chronic fatigue syndrome. J Med Food. 2002 Winter;5(4):211-20).

Beneficial effect in patients with fibromyalgia (Citera G, Arias M A, Maldonado-Cocco J A, Lazaro M A, Rosemffet M G, Brusco L I, Scheines E J, Cardinalli D P.: The effect of melatonin in patients with fibromyalgia: a pilot study. Clin Rheumatol. 2000;19(1):9-13).

Treatment of depression, anxiety, and sleep disorders (Cauffield J S, Forbes H J.: Dietary supplements used in the treatment of depression, anxiety, and sleep disorders. Lippincotts Prim Care Pract. May-June 1999;3(3):290-304).

Beneficial effects on psychiatric disorders (Miles A, Philbrick D R.: Melatonin and psychiatry. Biol Psychiatry. Feb. 15, 1988;23(4):405-25).

11. L-Carnitine/Acetyl_L-Carnitine

L-Carnitine has been described as a conditionally essential nutrient for humans. L-Carnitine is a betaine required for the transport of long-chain fatty acids into the mitochondria for fuel. It also facilitates the removal from the mitochondria of the excess short- and medium-chain fatty acids that accumulate during metabolism. L-Carnitine, and its acetyl derivative, acetyl-L-carnitine (ALCAR), affect other cellular functions, including maintaining key proteins and lipids of the mitochondria at sufficient levels and proper membrane orientation, for maximum energy production. ALCAR, like L-carnitine, is present in high concentration in the brain as well as muscle, and provides acetyl-equivalents for the production of the neurotransmitter acetylcholine. Experimental data have demonstrated an age-associated decrease of tissue levels of L-carnitine in animals, including humans, and an associated decrease in the integrity of the mitochondrial membrane. ALCAR is more widely used than L-carnitine in animal research and clinical trials to gain metabolic benefits to the brain, heart, liver, and other organs. Studies have shown that ALCAR has the following functions and effects on stress and stress-related disorders:

Anti-stress hormone glucocorticoid effects (McEwen B S, Spencer R L, Chapman S, Ganem J, O'Steen W K.: Neuroendocrine aspects of cerebral aging. Int J Clin Pharmacol Res. 1990;10(1-2):7-14).

Protecting effects on stress-induced gastric injury (Izgut-Uysal V N, Derin N, Agac A.: Protective effect of L-carnitine on gastric mucosal barrier in rats exposed to cold-restraint stress. Indian J Gastroenterol. July-August 2001;20(4):148-50).

Reducing stress-induced lipid peroxidation in rats (Izgut-Uysal V N, Agac A, Derin N. Effect of carnitine on stress-induced lipid peroxidation in rat gastric mucosa. J Gastroenterol. April 2001;36(4):231-6).

Protecting acute stress-induced injury by moderating dopamine output in mesocorticolimbic areas in rats (Tolu P, Masi F, Leggio B, Scheggi S, Tagliamonte A, De Montis M G, Gambarana C.: Effects of long-term acetyl-L-carnitine administration in rats: I. increased dopamine output in mesocorticolimbic areas and protection toward acute stress exposure. Neuropsychopharmacology. September 2002;27(3):410-20).

Protection against the disrupting effect of stress on the acquisition of appetitive behavior (Masi F, Leggio B, Nanni G, Scheggi S, De Montis M G, Tagliamonte A, Grappi S, Gambarana C.: Effects of long-term acetyl-L-carnitine administration in rats—II: Protection against the disrupting effect of stress on the acquisition of appetitive behavior. Neuropsychopharmacology. April 2003;28(4):683-93).

Improving spatial memory in old rats (Taglialatela G, Caprioli A, Giuliani A, Ghirardi O. Spatial memory and NGF levels in aged rats: natural variability and effects of acetyl-L-carnitine treatment. Exp Gerontol. September-October 1996;31(5):577-87).

Improving learning and reducing brain oxidation in old mice (Yasui F, Matsugo S, Ishibashi M, Kajita T, Ezashi Y, Oomura Y, Kojo S, Sasaki K.: Effects of chronic acetyl-L-carnitine treatment on brain lipid hydroperoxide level and passive avoidance learning in senescence-accelerated mice. Neurosci Lett. Dec. 16, 2002;334(3):177-80).

Enhancing learning capacity and cholinergic synaptic function in old rats (Ando S, Tadenuma T, Tanaka Y, Fukui F, Kobayashi S, Ohashi Y, Kawabata T.: Enhancement of learning capacity and cholinergic synaptic function by carnitine in aging rats. J Neurosci Res. Oct. 15, 2001;66(2):266-71).

Synergic effects with alpha-lipoic acid on improving memory in old rats (Liu J, Head E, Gharib A M, Yuan W, Ingersoll R T, Hagen T M, Cotman C W, Ames B N.: Memory loss in old rats is associated with brain mitochondrial decay and RNA/DNA oxidation: partial reversal by feeding acetyl-L-carnitine and/or R-alpha-lipoic acid. Proc Natl Acad Sci USA. Feb. 19, 2002;99(4):2356-61).

Inhibiting neonatal anoxia-induced memory deficit and the possible use for treating perinatal asphyctic insults in children (Dell'Anna E, Iuvone L, Calzolari S, Geloso M C. Effect of acetyl-L-carnitine on hyperactivity and spatial memory deficits of rats exposed to neonatal anoxia. Neurosci Lett. Feb. 28, 1997;223(3):201-5).lmproving spatial acquisition in a new environment in aged rats (Caprioli A, Markowska A L, Olton D S. Acetyl-L-Carnitine: chronic treatment improves spatial acquisition in a new environment in aged rats. J Gerontol A Biol Sci Med Sci. July 1995;50(4):B232-36).

Treating mild cognitive impairment and mild Alzheimer's disease (Montgomery S A, Thai L J, Amrein R.: Meta-analysis of double blind randomized controlled clinical trials of acetyl-L-carnitine versus placebo in the treatment of mild cognitive impairment and mild Alzheimer's disease. Int Clin Psychopharmacol. March 2003;18(2):61-71).

Treating mental decline in old humans (Salvioli G, Neri M. L-acetylcarnitine treatment of mental decline in the elderly. Drugs Exp Clin Res. 1994;20(4):169-76).

Treating Alzheimer's disease (Sano M, Bell K, Cote L, Dooneief G, Lawton A, Legler L, Marder K, Naini A, Stern Y, Mayeux R.: Double-blind parallel design pilot study of acetyl levocarnitine in patients with Alzheimer's disease. Arch Neurol. November 1992;49(11):1137-41)

Improving learning in old rats (Ghirardi O, Caprioli A, Milano S, Giuliani A, Ramacci M T, Angelucci L.: Active avoidance learning in old rats chronically treated with levocarnitine acetyl. Physiol Behav. July 1992;52(1): 185-7).

Improving memory in old rats (Ghirardi O, Giuliani A, Caprioli A, Ramacci M T, Angelucci L.: Spatial memory in aged rats: population heterogeneity and effect of levocarnitine acetyl. J Neurosci Res. February 1992;31 (2):375-9).

Imroving memory and learning in old rats (Barnes C A, Markowska A L, Ingram D K, Kametani H, Spangler E L, Lemken V J, Olton D S.: Acetyl-1-carnitine. 2: Effects on learning and memory performance of aged rats in simple and complex mazes. Neurobiol Aging. September-October 1990; 11(5):499-506).

Treating chronic fatigue syndrome patients (Vermeulen R C, Scholte H R.: Exploratory open label, randomized study of acetyl- and propionylcarnitine in chronic fatigue syndrome. Psychosom Med. March-April 2004;66(2):276-82).

Treating fatigue in multiple sclerosis patients (Tomassini V, Pozzilli C, Onesti E, Pasqualetti P, Marinelli F, Pisani A, Fieschi C.: Comparison of the effects of acetyl L-carnitine and amantadine for the treatment of fatigue in multiple sclerosis: results of a pilot, randomised, double-blind, crossover trial. J Neurol Sci. Mar. 15, 2004;218(1-2):103-8).

Reduced uptake in the brain linked to fatigue sensation (Kuratsune H, Yamaguti K, Lindh G, Evengard B, Hagberg G, Matsumura K, Iwase M, Onoe H, Takahashi M, Machii T, Kanakura Y, Kitani T, Langstrom B, Watanabe Y. Brain regions involved in fatigue sensation: reduced acetylcarnitine uptake into the brain. Neuroimage. November 2002;17(3):1256-65).

12. Coenzyme Q10 (CoQ)

Coenzyme Q10 (CoQ) belongs to a family of compounds known as ubiquinones. All animals, including humans, can synthesize ubiquinones but old animals may have a decreased ability for synthsis because there is a universal decrease in all organs of humans. CoQ plays an essential role in mitochondrial electron chain transfer for mitochondrial ATP synthesis. Studies have demonstrated the following functions and effects on stress and stress-related disorders:

Antioxidant functions (Ernster L, Dallner G. Biochemical, physiological and medical aspects of ubiquinone function. Biochim Biophys Acta. 1995;1271(1):195-204; Ernster L: Ubiquinol as a biological antioxidnat: a review. In Oxidative Processes and Antioxidants (eds. R. Paoletti et al.) pp 185-98, 1994)

Improving Parkinson's disease symptoms (Shults C W, Oakes D, Kieburtz K, et al. Effects of coenzyme Q10 in early Parkinson disease: evidence of slowing of the functional decline. Arch Neurol. 2002;59(10):1541-1550).

Improving Huntington's disease (Beal M F. Coenzyme Q10 as a possible treatment for neurodegenerative diseases. Free Radic Res. 2002;36(4):455-460; Koroshetz W J, Jenkins B G, Rosen B R, Beal M F. Energy metabolism defects in Huntington's disease and effects of coenzyme Q10. Ann Neurol. 1997;41 (2):160-165)

Beneficial as an adjunct to conventional therapy for breast cancer (Hodges S, Hertz N, Lockwood K, Lister R. CoQ10: could it have a role in cancer management? Biofactors. 1999;9(2-4):365-370).

Beneficial in the treatment of hypertension (Langsjoen P H, Langsjoen A M. Overview of the use of CoQ10 in cardiovascular disease. Biofactors. 1999;9(2-4):273-284)

Improving endothelium-dependent vasodilation in diabetic patients with abnormal serum lipid profiles (Watts G F, Playford D A, Croft K D, Ward N C, Mori T A, Burke V. Coenzyme Q(10) improves endothelial dysfunction of the brachial artery in Type II diabetes mellitus. Diabetologia. 2002;45(3):420-426)

Inhibiting the formation of atherosclerotic lesions (Witting P K, Pettersson K, Letters J, Stocker R. Anti-atherogenic effect of coenzyme Q10 in apolipoprotein E gene knockout mice. Free Radic Biol Med. 2000;29(3-4):295-305).

Improvement mitochondrial encephalomyopathies in humans (Shoffner J M. Oxidative phosphorylation diseases. In: Scriver C R, Beaudet A L, Sly W S, Valle D, eds. The metabolic and molecular bases of inherited disease. Vol 2. 8th ed. New York: McGraw-Hill; 2001:2367-2392).

Improving insulin secretion and preventing progressive hearing loss in patients with maternally inherited diabetes mellitus and deafness, which is result of a mutation in mitochondrial DNA (Suzuki S, Hinokio Y, Ohtomo M, et al. The effects of coenzyme Q10 treatment on maternally inherited diabetes mellitus and deafness, and mitochondrial DNA 3243 (A to G) mutation. Diabetologia. 1998;41(5):584-588; Alcolado J C, Laji K, Gill-Randall R. Maternal transmission of diabetes. Diabet Med. 2002;19(2):89-98)

13. Choline

Choline is an essential nutrient. It can be manufactured in the body (from the amino acid methionine), although there is some debate whether it can be made in sufficient amounts for optimal health. Folic acid and vitamin B12 are also needed to process choline. Choline plays a role in brain development (as an amine precursor for the neurotransmitter acetylcholine), liver function and cardiovascular health.

Enhances memory and repairs memory damage: Prenatal deficiency causes while sufficiency prevents age-associated memory decay (Jan Krzysztof Blusztajn: Choline, a Vital Amine. Science. Aug. 7, 1998;281 (5378):794-5; Yang Y, Liu Z, Cermak J M, Tandon P, Sarkisian M R, Stafstrom C E, Neill J C, Blusztajn J K, Holmes G L. Protective effects of prenatal choline supplementation on seizure-induced memory impairment. J Neurosci. Nov. 15, 2000;20(22):RC109).

Reducing epilepsy-associated memory impairment (Holmes G L, Yang Y, Liu Z, Cermak J M, Sarkisian M R, Stafstrom C E, Neill J C, Blusztajn J K. Seizure-induced memory impairment is reduced by choline supplementation before or after status epilepticus. Epilepsy Res. January 2002;48(1-2):3-13).

Improves Alzheimer's disease (Cacabelos R, Caamano J, Gomez M J, Fernandez-Novoa L, Franco-Maside A, Alvarez X A. Therapeutic effects of CDP-choline in Alzheimer's disease. Cognition, brain mapping, cerebrovascular hemodynamics, and immune factors. Ann N Y Acad Sci. Jan. 17, 1996;777:399-403; Franco-Maside A, Caamano J, Gomez M J, Cacabelos R. Brain mapping activity and mental performance after chronic treatment with CDP-choline in Alzheimer's disease. Methods Find Exp Clin Pharmacol. October 1994;16(8):597-607; Caamano J, Gomez M J, Franco A, Cacabelos R.: Effects of CDP-choline on cognition and cerebral hemodynamics in patients with Alzheimer's disease. Methods Find Exp Clin Pharmacol. April 1994;16(3):211-8).

Improveing Parkinson's disease (Cubells J M, Hernando C. Clinical trial on the use of cytidine diphosphate choline in Parkinson's disease. Clin Ther. 1988;10(6):664-71.; Agnoli A, Ruggieri S, Denaro A, Bruno G. New strategies in the management of Parkinson's disease: a biological approach using a phospholipid precursor (CDP-choline). Neuropsychobiology. 1982;8(6):289-96.

Important for brain development, liver function and carcinogenesis (Zeisel S H. Choline: an important nutrient in brain development, liver function and carcinogenesis. J Am Coll Nutr. October 1992;11(5):473-81).

Combination with carnitine and caffeine has the same function as exercise: loss of body fat (Hongu N, Sachan D S. Caffeine, carnitine and choline supplementation of rats decreases body fat and serum leptin concentration as does exercise. J Nutr. February 2000;130(2):152-7; Sachan D S, Hongu N. Increases in VO(2)max and metabolic markers of fat oxidation by caffeine, carnitine, and choline supplementation in rats. J Nutr Biochem. October 2000;11(10):521-6; Hongu N, Sachan D S.: Carnitine and choline supplementation with exercise alter carnitine profiles, biochemical markers of fat metabolism and serum leptin concentration in healthy women. J Nutr. January 2003;133(1):84-9).

14. Creatine

Creatine is an amino acid, which is made in the body by the liver and kidneys, and is derived from the diet through meat and animal products. In the body, creatine is changed into a molecule called “phosphocreatine” which serves as a storage reservoir for quick energy. Phosphocreatine is especially important in tissues such as the voluntary muscles and the nervous system, which periodically require large amounts of energy.

Improving brain performance in humans (Rae C, Digney A L, McEwan S R, Bates T C.: Oral creatine monohydrate supplementation improves brain performance: a double-blind, placebo-controlled, cross-over trial. Proc R Soc Lond B Biol Sci. Oct. 22, 2003;270(1529):2147-50; Greenhaff P L. Creatine and its application as an ergogenic aid. Int J Sport Nutr. June 1995;5 Suppl:S100-10.

Enhancing energy synthesis (Kurosawa Y, Hamaoka T, Katsumura T, Kuwamori M, Kimura N, Sako T, Chance B.: Creatine supplementation enhances anaerobic ATP synthesis during a single 10 sec maximal handgrip exercise. Mol Cell Biochem. February 2003;244(1-2):105-12; Casey A, Greenhaff P L. Does dietary creatine supplementation play a role in skeletal muscle metabolism and performance? Am J Clin Nutr. August 2000;72(2 Suppl):607S-17S; Greenhaff P L, Bodin K, Soderlund K, Hultman E. Effect of oral creatine supplementation on skeletal muscle phosphocreatine resynthesis. Am J Physiol. May 1994;266(5 Pt 1):E725-30).

Improving human strength (Dempsey R L, Mazzone M F, Meurer L N.: Does oral creatine supplementation improve strength? A meta-analysis. J Fam Pract. November 2002;51(11):945-51; Kraemer W J, Volek J S.: Creatine supplementation. Its role in human performance. Clin Sports Med. July 1999; 18(3):651-66),

Neuroprotective effect on cerebral ischemia damage in mice (Zhu S, Li M, Figueroa B E, Liu A, Stavrovskaya I G, Pasinelli P, Beal M F, Brown R H Jr, Kristal B S, Ferrante R J, Friedlander R M.: Prophylactic creatine administration mediates neuroprotection in cerebral ischemia in mice. J Neurosci. Jun. 30, 2004;24(26):5909-12).

Neuroprotective effects on NMDA and malonate toxicity (Malcon C, Kaddurah-Daouk R, Beal M F. Neuroprotective effects of creatine administration against NMDA and malonate toxicity. Brain Res. Mar. 31, 2000;860(1-2):195-8).

Improving mitochondrial encephalomyopathies (Komura K, Hobbiebrunken E, Wilichowski E K, Hanefeld F A.: Effectiveness of creatine monohydrate in mitochondrial encephalomyopathies. Pediatr Neurol. January 2003;28(1):53-8).

Facilitating rehabilitation of disuse atrophy (Hespel P, Op't Eijnde B, Van Leemputte M, Urso B, Greenhaff P L, Labarque V, Dymarkowski S, Van Hecke P, Richter E A.: Oral creatine supplementation facilitates the rehabilitation of disuse atrophy and alters the expression of muscle myogenic factors in humans. J Physiol. Oct. 15, 2001;536(Pt 2):625-33).

Beneficial to diabetes (Op't Eijnde B, Urso B, Richter E A, Greenhaff P L, Hespel P.: Effect of oral creatine supplementation on human muscle GLUT4 protein content after immobilization. Diabetes. January 2001;50(1):18-23).

Augmenting skeletal muscle endurance and attenuates in chronic heart failure (Andrews R, Greenhaff P, Curtis S, Perry A, Cowley A J.: The effect of dietary creatine supplementation on skeletal muscle metabolism in congestive heart failure. Eur Heart J. April 1998;19(4):617-22).

Neuroprotective effects on amyotrophic lateral sclerosis. (Klivenyi P, Ferrante R J, Matthews R T, Bogdanov M B, Klein A M, Andreassen O A, Mueller G, Wermer M, Kaddurah-Daouk R, Beal M F.: Neuroprotective effects of creatine in a transgenic animal model of amyotrophic lateral sclerosis. Nat Med. March 1999;5(3):347-50; Klivenyi P, Kiaei M, Gardian G, Calingasan N Y, Beal M F.: Additive neuroprotective effects of creatine and cyclooxygenase 2 inhibitors in a transgenic mouse model of amyotrophic lateral sclerosis. J Neurochem. February 2004;88(3):576-82).

Neuroprotective effect on Parkinson's disease (Klivenyi P, Gardian G, Calingasan N Y, Yang L, Beal M F.: Additive neuroprotective effects of creatine and a cyclooxygenase 2 inhibitor against dopamine depletion in the 1-methyl-4-phenyl-1,2,3,6-tetrahydropyridine (MPTP) mouse model of Parkinson's disease. J Mol Neurosci. 2003;21 (3):191-8; Matthews R T, Ferrante R J, Klivenyi P, Yang L, Klein A M, Mueller G, Kaddurah-Daouk R, Beal M F.: Creatine and cyclocreatine attenuate MPTP neurotoxicity. Exp Neurol. May 1999;157(1):142-9).

Neuroprotective effect on Huntington's disease (Dedeoglu A, Kubilus J K, Yang L, Ferrante K L, Hersch S M, Beal M F, Ferrante R J.: Creatine therapy provides neuroprotection after onset of clinical symptoms in Huntington's disease transgenic mice. J Neurochem. June 2003;85(6):1359-67; Ferrante R J, Andreassen O A, Jenkins B G, Dedeoglu A, Kuemmerle S, Kubilus J K, Kaddurah-Daouk R, Hersch S M, Beal M F.: Neuroprotective effects of creatine in a transgenic mouse model of Huntington's disease. J Neurosci. Jun. 15, 2000;20(12):4389-97; Andreassen O A, Dedeoglu A, Ferrante R J, Jenkins B G, Ferrante K L, Thomas M, Friedlich A, Browne S E, Schilling G, Borchelt D R, Hersch S M, Ross C A, Beal M F.: Creatine increase survival and delays motor symptoms in a transgenic animal model of Huntington's disease. Neurobiol Dis. June 2001;8(3):479-91; Matthews R T, Yang L, Jenkins B G, Ferrante R J, Rosen B R, Kaddurah-Daouk R, Beal M F.: Neuroprotective effects of creatine and cyclocreatine in animal models of Huntington's disease. J Neurosci. Jan. 1, 1998; 18(1): 56-63.

15. Review Papers on the Functions and Effects of the Ingredients of this Application

The afore cited are specific research papers on individual components of the ingredients containing in this application, the following are some review papers include more than one of the ingredients of this application on stress and stress-related disorders.

The anti-stress effects of nutritional and botanical substances—such as adaptogenic herbs, specific vitamins including ascorbic acid, vitamins B1 and B6, the coenzyme forms of vitamin B5 (pantethine) and B12 (methylcobalamin), the amino acid tyrosine, and other nutrients such as lipoic acid, phosphatidylserine, and plant sterol/sterolin combinations (Kelly G S.: Nutritional and botanical interventions to assist with the adaptation to stress. Altern Med Rev. August 1999;4(4):249-65.)

The anti-stress and delaying aging agents including natural products (alpha-tocopherol, cysteine, carnitine, acetyl-L-carnitine, uric acid, SOD, glutathione, DHEA-dehydroepiandrosterone, Yizhiyishou-an extract of traditional Chinese herbs, Manda-a fermented natural food) and other synthetic substances (butylated hydroxytoluene, 2-mercaptoethylamine, hydroxylamine, N-tert-alpha-phenyl-butynitrone, L-deprenyl, ionol, dimethylsulfoxide, desferrioxamine) (Liu J, Mori A.: Stress, aging, and brain oxidative damage. Neurochem Res. November 1999;24(11):1479-97).

Phosphatidylserine (PS), acetyl-l-carnitine (ALC), vinpocetine, Ginkgo biloba extract (GbE), and Bacopa monniera (Bacopa) for preventing and treating cognitive dysfunction (Kidd P M.: A review of nutrients and botanicals in the integrative management of cognitive dysfunction. Altern Med Rev. June 1999;4(3):144-61)

Neuroprotective effects of natural and nutritional anti-stress antioxidants, including vitamins A, C, E, estrogen, dehydroepiandrosterone, glutathione, acetyl-carnitine, and some herbal extracts (Jiankang Liu* and Akitane Mori**Oxidative Damage Hypothesis of stress-associated aging acceleration: neuroprotective effects of natural and nutritional antioxidants. Res. Commun Brain & Nerve (in press) 2005).

The identification and classification of mitochondrial cofactors and nutrients: vitamins, antioxidants, energy enhancers, etc to delay and treat cognitive dysfunction, Alzheimer's and Parkinson's disease (aiankang Liu and Bruce N. Ames.; Reducing mitochondrial decay with mitochondrial nutrients to delay and treat cognitive dysfunction, Alzheimer's and Parkinson's disease Nutritional Neuroscience (in press), 2005).

Brain-specific nutrients such as vitamin B complex, vitamin E, ubiquinone, ginkgo biloba, and phosphatidylserine, and stress-relieving meditation, mind-body and cognitive exercise, antiaging drugs like L-deprenyl citrate, as well as hormones such as dehydroepiandrosterone and pregnenolone for preventing memory loss (Khalsa D S.: Integrated medicine and the prevention and reversal of memory loss. Altern Ther Health Med. November 1998;4(6):38-43.)

Coenzyme Q(10) (ubiquinone); other antioxidants such as ascorbic acid, vitamin E, and lipoic acid; riboflavin; thiamin; niacin; vitamin K (phylloquinone and menadione); creatine; and carnitine for treating mitochondrial disorders (Marriage B, Clandinin M T, Glerum D M.: Nutritional cofactor treatment in mitochondrial disorders. J Am Diet Assoc. August 2003;103(8):1029-38).

Glutathione and other SH-containing antioxidants, vitamins, and polyphenolic compounds for preventing and treating Alzheimer's disease (Butterfield D, Castegna A, Pocernich C, Drake J, Scapagnini G, Calabrese V.: Nutritional approaches to combat oxidative stress in Alzheimer's disease. J Nutr Biochem. August 2002;13(8):444).

Creatine, coenzyme Q(10), Ginkgo biloba, nicotinamide, and acetyl-L-carnitine for preventing and treating Parkinson's disease (Beal M F.: Bioenergetic approaches for neuroprotection in Parkinson's disease. Ann Neurol. 2003;53 Suppl 3:S39-47; discussion S47-8).

The age-delaying effects and preventing and ameliorating effects on age related diseases by aqueous and lipid soluble cellular compartments (Adenosine Triphosphate (ATP), S-Adenosylmethione (SAMe), Ubiquinone/Coenzyme Q10 (Co-Q10). Thiamine pyrophosphate (TPP), Flavin mononucleotide (FMN) & flavin adenine dinucleotide (FAD), Nicotinamide adenine dinucleotide (NAD+/NADH) & nicotinamide adenine dinucleotide phosphate (NADP+/NADPH), Coenzyme A (CoA) and phosphopantetheine (part of the acyl carrier protein ACP), Pyridoxal-5-phosphate (PLP), Biotin, Tetrahydofolates: 6 inter-convertible foyl coenzymes, Tetrahydrobiopterin, Adenosylcobalamin, Methylcobalamin, Vitamin K, Uridine diphosphate (UDP) glucose, Uridine diphosphate (UDP) glucuoronic acid, Cytidine diphosphate-choline/ethanolamine/diacylglycerol, Carnitine, Lipoamide (lipoate)) (Price, M C. Longevity Report 91, www.quantium.cwc.net/lr91.html, 2001).

16. Different Combinations of the Ingredients of this Application having Adding or Synergistic Effects

Anti-stress effect of mixture of vitamin C, Vitamin E, beta-carotene, selenium, and zinc for anti-stress (Ushakova T, Melkonyan H, Nikonova L, Mudrik N, Gogvadze V, Zhukova A, Gaziev A I, Bradbury R.: The effect of dietary supplements on gene expression in mice tissues. Free Radic Biol Med. 1996;20(3):279-84)

Resisting stress effects of Vitamin A, C, E and C+E ( Zaidi S M, Al-Qirim T M, Hoda N, Banu N.: Modulation of restraint stress induced oxidative changes in rats by antioxidant vitamins. J Nutr Biochem. November 2003; 14(11):633-6

Anti-stress effect of Vitamin C+thiamine+pyrodoxine (Shelygina N M, Spivak RIa, Zaretskii M M, Panichkina V I, Gusiatinskaia V M.: Influence of vitamins C, BI, and B6 on the diurnal periodicity of the glucocorticoid function of the adrenal cortex in patients with atherosclerotic cardiosclerosis. Vopr Pitan. March-April 1975(2):25-9)

Vitamins E, A, B-6, and B-12 for improving memory in old people (La Rue A, Koehler K M, Wayne S J, Chiulli S J, Haaland K Y, Garry P J.: Nutritional status and cognitive functioning in a normally aging sample: a 6-y reassessment. Am J Clin Nutr. January 1997;65(1):20-9.).

Vtiamin E+alpha-lipoic acid+coenzyme Q+carnitine+selenomethione for enhancing antioxidant defense system (Mosca L, Marcellini S, Perluigi M, Mastroiacovo P, Moretti S, Famularo G, Peluso I, Santini G, De Simone C.: Modulation of apoptosis and improved redox metabolism with the use of a new antioxidant formula. Biochem Pharmacol. Apr. 1, 2002;63(7):1305-14).

alpha-Tocopherol acetate+L-carnitine+alpha-lipoic acid+ascorbic acid+spinach flakes+tomato pomace+grape pomace, carrot granules+citrus pulp for reversal of age-associated learning and memory decline (Milgram N W, Head E, Zicker S C, Ikeda-Douglas C J, Murphey H, Muggenburg B, Siwak C, Tapp D, Cotman C W.: Learning ability in aged beagle dogs is preserved by behavioral enrichment and dietary fortification: a two-year longitudinal study. Neurobiol Aging. January 2005;26(1):77-90)

Synergy of Cofactors: RNA+pantothenate+pyrodoxine+biotin and RNA+pyrodoxine+biotin for increasing lifespan (Price, M C. Longevity Report 91, www.quantium.cwc.net/lr91.html, 2001).

Riboflavin+L-carnitine; Riboflavin+niacin; Vitamin K3+ascorbic acid; CoQ+vitamin K3+ascorbic acid+thiamine+riboflavin+niacin; L-carnitine+choline+caffeine; Lipoic acid+carnitine/acetyl-L-carnitine for treating mitochondrial disorders (Liu, J and Ames, B N.: Reducing mitochondrial decay with mitochondrial nutrients to delay and treat cognitive dysfunction, Alzheimer's and Parkinson's disease. Nutr. Neurosci. (in press), 2005)

Vitamin E+ginko biloba_pycnogenol+ascorbyl palmitate; vitamin E+phosphatidyl choline+pyruvate for increasing lifesapn, reducing brain pathology, reducing DNA damage, treating cognitive dysfunction and Alzheimer's disease (Liu, J and Ames, B N.: Reducing mitochondrial decay with mitochondrial nutrients to delay and treat cognitive dysfunction, Alzheimer's and Parkinson's disease. Nutr. Neurosci. in press, 2005).

Vitamin E+vitamin C for synergistic antioxidant effects on Alzheimer's disease (AD) and AD with vascular dementia or cerebrovascular disease (Liu, J and Ames, B N.: Reducing mitochondrial decay with mitochondrial nutrients to delay and treat cognitive dysfunction, Alzheimer's and Parkinson's disease. Nutr. Neurosci. (in press), 2005)

Acetyl-L-carnitine+donepezil+rivastigmine for treating Alzheimer's disease (AD) (Liu, J and Ames, B N.: Reducing mitochondrial decay with mitochondrial nutrients to delay and treat cognitive dysfunction, Alzheimer's and Parkinson's disease. Nutr. Neurosci. (in press), 2005)

Lipoic acid+CoQ for synergistic antioxidant effects on age-associated cognitive dysfunction, Alzheimer's and Parkinson's disease (Liu, J and Ames, B N.: Reducing mitochondrial decay with mitochondrial nutrients to delay and treat cognitive dysfunction, Alzheimer's and Parkinson's disease. Nutr. Neurosci. (in press), 2005)

CoQ+vitamin E is more effective in inhibiting atherosclerosis. Because CoQ is capable of regenerating α-tocopherol, therefore, cosupplementation of apolipoprotein E gene knockout mice with α-tocopherol and coenzyme Q10 was found to be more effective in inhibiting atherosclerosis than supplementation with either α-tocopherol or coenzyme Q10 alone (Thomas S R, Leichtweis S B, Pettersson K, et al. Dietary cosupplementation with vitamin E and coenzyme Q(10) inhibits atherosclerosis in apolipoprotein E gene knockout mice. Arterioscler Thromb Vasc Biol. 2001;21 (4):585-593)

CoQ+ramacemide; CoQ+ramacemide+transglutamenase inhibitor+nitric oxide synthase inhibitor for treating Huntington's disease (Beal, M F.: Bioenergetic approaches for neuroprotection in Parkinson's disease. Ann Neurolo 53(suppl):S39-S48, 2003)

CoQ+vitamin K3+ascorbate+methylprednisolone for treating mitochondrial disorders (Stanley C A.: New genetic defects in mitochondrial fatty acid oxidation and carnitine deficiency. Adv Pediatr. 1987;34:59-88).

Acetyl-L-carnitine+alpha-lipoic acid for improving memory and increasing antioxidant defense system (Liu J, Head E, Gharib A M, Yuan W, Ingersoll R T, Hagen T M, Cotman C W, Ames B N.: Memory loss in old rats is associated with brain mitochondrial decay and RNA/DNA oxidation: partial reversal by feeding acetyl-L-carnitine and/or R-alpha-lipoic acid. Proc Natl Acad Sci USA. Feb. 19, 2002;99(4):2356-61).

Melatonin synergizes ascorbic acid and lipoic acid for protecting DNA damage (Lopez-Burillo S, Tan D X, Mayo J C, Sainz R M, Manchester L C, Reiter R J.: Melatonin, xanthurenic acid, resveratrol, EGCG, vitamin C and alpha-lipoic acid differentially reduce oxidative DNA damage induced by Fenton reagents: a study of their individual and synergistic actions. J Pineal Res. May 2003;34(4):269-77).

Effect on reactive oxygen and nitrogen species, memory, learning, aging and neuropathology by a combination of the following ingredients: thiamine, niacin, pyrodoxine, vitamin B12, vitamin C, vitamin D, vitamin E, acetyl-L-carnitine, alpha-lipoic acid, beta-carotene, chromium picolinate, coQ, DHEA, folic acidglutathione, magnesium, melatonin, N-acetylcysteine, potassium, rutin, selenium, zinc, and some commercial products or plant extracts, including ASA of Lifebrand, cod liver oil, flax seed iol, , garlic, ginger, ginko biloba, ginseng, green tea extracts (Lemon J A, Boreham D R, Rollo C D.: A dietary supplement abolishes age-related cognitive decline in transgenic mice expressing elevated free radical processes. Exp Biol Med (Maywood). July 2003;228(7):800-10.)

Effect on extending lifespan in rats by the same combination in 17) (Jennifer A Lemon1,3, Douglas R Boreham2, C David Rollo.: A Complex Dietary Supplement Extends Longevity of Mice J. Gerontology 60A: 275-9, 2005).

The doses of the ingredients have been based on the dietary reference intakes (DRIs, recommended dietary allowance RDA), the supraphysiological doses used in clinical trials (for those which have RDA), (Table 1) and the commonly clinical used doses (for those which do not have RDA) (Table 2). The amounts of each ingredients for each specified method is composed according to the specific aim according the chemical properties, biological function, adding or synergistic actions of the ingredients used. Since the important concept is to have a mixture not only with different functions, but also with adding and the synergistic effects for optimum effects, the amounts used will be much smaller than that used singly.

TABLE 1
Lists the dietary reference intakes (DRIs, recommended dietary
allowance RDA or Adequate Intake (AI)) and the supraphysiological
doses used in clinical trials. The doses of each nutrients
in Nutrigen are based on these clinical data.

| | | | Supra- | |
|---|---|---|---|---|
| | | | physiological | |
| | | | doses | |
| | | DRI | clinically | |
| | | (Men/Women) | used | |
| B-vitamins | Thiamine (B1) | 1.2 mg/1.1 mg | 1000 | mg |
| | Niacin (B3) | 16 mg/14 mg | 2000 | mg |
| | Pantothenate | 5 mg/5 mg | 150 | mg |
| | (B5) | | | |
| | Pyridoxine (B6) | 1.3 mg/1.3 mg | 1000 | mg |
| | Folic acid | 400 μg/400 μg | 40,000 | μg |
| | Riboflavin (B2) | 1.3 mg/1.1 mg | 400 | mg |
| Other | Ascorbic acid | 90 mg/75 mg | 10,000 | mg |
| vitamins | (Vitamin C) | | | |
| | Alpha-, gamma- | 15 mg/15 mg | 800 | mg |
| | Tocopherols | | | |
| | (Vitamin E) | | | |
| | Vitamin A | 900 μg/700 μg | 10,000 | μg |
| | (retinol) | | | |
| | Vitamin D | 10 μg/10 μg | 250-1250 | μg |
| | Vitamin K | 120 μg/90 μg | 1000 | μg |

TABLE 2
The clinically used doses of other components (Recommended
dietary allowance (RDA) not available)

| | Dose range used | |
|---|---|---|
| Substance | clinically | |
| R-Alpha-Lipoic acid (LA)/ | 300-1,000 | mg |
| N-Acetyl-cysteine | 3,000 | mg |
| L-Carnosine | 800 | mg |
| Tyrosine | 6,000-9,000 | mg |
| Vanillin | 0.5 | g/kg ketchup |
| Phosphatidylserine | 50-800 | mg |
| Resveratrol | 25 | mg |
| Dehydroepiandrosterone (DHEA) | 25-50 | mg |
| Melatonin | 0.1-3 | mg |
| CoQ10 | 10-1,200 | mg |
| Acetyl-L-carnitine | 100-2,000 | mg |
| Choline | 50-1000 | mg |
| Creatine | 100-20,000 | mg |

For optimal utilization of these mitochondrial nutrients, they should be administered with energy source (or carbohydrate source), which can be a variety of known substances. Example include a natural non-citrus fruit drink, such as grape juice or apple juice, such that it is sufficient to provide between about 50 to 150 calories.

A preferred formulation provides the mitochondrial nutrients in a timed-release formulation to provide a steady supply of nutrients to the mitochondria that work 24 hours a day. One method of accomplishing timed release is chemically combining the mitochondrial nutrients with other molecules, which can slow the process of making the mitochondrial nutrients available. Also the use of different salts of the mitochondrial nutrients with different dissolution rates provides for the desired gradual release of each mitochondrial nutrients.

In addition, two other basic systems are used to control chemical release: coating a core comprising the mitochondrial nutrients and excipients (coated system) and incorporating the mitochondrial nutrients into a matrix (matrix system).

### V. EXAMPLE

An example of a dietary supplement for reliving stress, delaying aging, preventing and improving stress-/age-related disorders and diseases will have the following aspects and ingredients (for b.i.d):

| | Amount (mg except |
|---|---|
| Ingridents | those specified) |
| Cyanocobalamin | 20 ug |
| Thiamin | 4 |
| Niacin | 50 |
| Pyridoxine | 5 |
| Pantothenate | 20 |
| Folic acid | 0.8 |
| alpha-Tocopherol | 30 |
| Ascorbic acid | 60 |
| Calcium | 246 |
| Vitamin A | 500 IU |
| alpha-Lipoic acid | 100 |
| N-Acetyl cysteine | 100 |
| L-Carnosine | 100 |
| Tyrosine | 100 |
| Vaniline | 20 |
| Phosphatidylserine | 10 |
| Resveratrol | 10 |
| DHEA | 5 |
| Melatonin | 1 |

What is claimed is:

1. A method for relieving stress, preventing and improving stress-related disorders comprising orally use of a composition containing mitochondrial nutrients.

2. A method according to claim 1 wherein the stress includes all stress-related abnormalities in physiology, psychology, and behavior for every age group experienced in working, studying, and daily life. Psychological abnormalities include anxiety, loneliness, boredom, helplessness, aggressiveness, restlessness, over-reaction, loss of motivation; Physiological abnormalities includes tenseness, stiff neck, tired muscle, flushing of skin, chronic indigestion, dizziness, stomachaches, cold sweats; Behavioral abnormalities include impatience, carelessness, sighing, unable to laugh, eating disorder, forgetfulness, sleeplessness, and reduced social communication. Stress-related disorders include aging acceleration, ulcer, cancer, type II diabetes, obesity, hypertension, osteoporosis, depression, glaucoma, cataracts, Alzheimer's disease, Parkinson's disease, and cardiovascular disease.

3. A method according to claim 1 wherein the mitochondrial nutrients are cyanocobalamin, thiamin, niacin, pyridoxine, pantothenate, folic acid, alpha-tocopherol, ascorbic acid, calcium, vitamin A, alpha-lipoic acid, N-acetyl cysteine, L-carnosine, tyrosine, vanillin, phosphatidylserine, resveratrol, dehydroepiandrosterone, and melatonin.

4. A method according to claim 1 wherein the composition comprises cyanocobalamin 2-1,000 ug, thiamin 1-1,000 mg, niacin 15-2,000 mg, pyridoxine 1-1,000 mg, pantothenate 5-150 mg, folic acid 400-40,000 ug, alpha-tocopherol 10-800 mg, ascorbic acid 50-10,000 mg, calcium 20-2,000mg, vitamin A 200-10,000 ug, alpha-lipoic acid 100-1,000 mg, N-acetyl cysteine 100-3,000 mg, L-carnosine 100-9,000 mg, tyrosine 100-9,000 mg, vanillin 10-100 mg, phosphatidylserine 10-800 mg, resveratrol 10-50 mg, dehydroepiandrosterone 1-50 mg, and melatonin 0.1-3 mg. The amounts of each ingredients for each specified method is composed according to the specific aim according the chemical properties, biological function, adding or synergistic actions of the ingredients used. Since the important concept is to have a mixture not only with different functions, but also with adding and the synergistic effects for optimum effects, the amounts used will be much smaller than that used singly.

5. A method according to claim 1 where the composition further contains an active ingredient selected from the group of mitochondrial nutrients consisting of coenzyme Q, acetyl-L-carnitine, choline, and creatine.

6. A method according to claim 4 wherein the composition comprises coenzyme Q 10-200 mg, acetyl-L-carnitine 100-2,000 mg, choline 50-1,000 mg, and creatine 100-2,000 mg.
Page 48 of 49
